(19) United States
(12) Patent Application Publication
(10) Pub. No.: US 2022/0351810 A1
(43) Pub. Date: Nov. 3, 2022

(54) DIRECT DATA CONNECTION FOR UNIVERSAL COLLECTION OF HEALTH DATA

(71) Applicant: GENENTECH, INC., South San Francisco, CA (US)

(72) Inventors: Mehek MOHAN, San Francisco, CA (US); Abdi ODAY, South San Francisco, CA (US); John A. JONES, Manalapan, NJ (US)

(73) Assignee: Genetech, Inc., South San Francisco, CA (US)

(21) Appl. No.: 17/732,332

(22) Filed: Apr. 28, 2022

Related U.S. Application Data
(60) Provisional application No. 63/182,221, filed on Apr. 30, 2021.

(51) Int. Cl. G16H 10/60

(52) U.S. Cl. CPC G16H 10/60

(57) ABSTRACT

The present disclosure relates to techniques for direct data connection (DDC) for universal collection of clinical trial health data. Particularly, aspects are directed to obtaining, by an application programming interface of a DDC system, health data for a subject from a site. The DDC system determines a visit status of the subject associated with the health data. The DDC system determines a form for the subject that is to be completed for capturing the health data. The DDC system maps data elements of the health data in the site specific format to data elements of the form in an electronic data capture format and establishes instructions for translating the data elements to the electronic data capture format. The DDC system translates the data elements based on the instructions and communicates information to an electronic data capture system.

### CROSS-REFERENCE TO RELATED APPLICATIONS

This application claims the benefit of and priority to U.S. Provisional Patent Application Nos.: 63/182,221 (filed on Apr. 30, 2021), which is hereby incorporated by reference in its entirety for all purposes.

### FIELD

The present disclosure relates to health data collection, and in particular to a direct data connection for universal collection of health data.

### BACKGROUND

In healthcare, clinical trials help answer questions related to medical treatments and interventions. For example, clinical trials for new drug treatments determine efficacy, safety, dosage, side effects, and other information related to using a drug treatment. Subjects enroll in clinical health trials and have health data collected throughout the clinical trial that is analyzed to determine the outcomes. In order to support clinical trial research coordinators, software developers have looked to digitize the collection of the health data from the subjects. While digitizing the collection of data streamlines portions of the process, other unique challenges are created as a result. For example, each system used to collect health data may use its own format for storing the health data.

### SUMMARY

In various embodiments, a computer-implemented method is provided that involves obtaining, by an application programming interface (API) of a direct data connection (DDC) system, health data for a subject from a site (e.g., an electronic health record (EHR) system, a clinical trial management system (CTMS), data warehouse, laboratory information management system (LIMS), etc.). The site can be an end point of the API and the health data can be obtained in a site specific format. The method also involves determining, by the DDC system, a visit status of the subject associated with the health data. The visit status identifies whether the health data for the subject was collected in accordance with a scheduled visit or an unscheduled visit. The method additionally involves determining, by the DDC system, a form for the subject that is to be completed for capturing the health data based on the health data associated with the subject the visit status determined for the subject. For example, the form can be a urinalysis form, a hematology form, a chemistry form, or any other suitable form. The method also involves mapping data elements of the health data in the site specific format to counterpart data elements of the form in an electronic data capture format and establishing, by the DDC system, instructions for how the data elements of the health data in the site specific format are to be translated to the data elements of the health data in the electronic data capture format based on the mappings. The DDC system translates the data elements of the health data in the site specific format to the data elements of the health data in the electronic data capture format based on the instructions and communicates the visit status of the subject, information identifying the form for the subject, and the data elements of the health data in the electronic data capture format, to an electronic data capture system.

In some embodiments, the health data is obtained from a plurality of systems at the sites including an EHR system and a supplementary health data system (e.g., CTMS or data warehouse), and the health data is obtained in a plurality of different site specific formats.

In some embodiments, the health data is obtained by the site making a call using the API to a server associated with the DDC system.

In some embodiments, determining the visit status involves identifying, by the DDC system, an actual visit date on which the health data was collected and identifying, by the DDC system, whether a scheduled visit date exists for the subject on the actual visit date. Determining the visit status also involves, in response to identifying the scheduled visit date exists for the subject on the actual visit date, determining the visit status as a scheduled visit and in response to identifying the scheduled visit date does not exist for the subject on the actual visit date, determining the visit status as an unscheduled visit.

In some embodiments, determining the form comprises involves identifying, by the DDC system, types of data within the health data and identifying, by the DDC system, a visit alignment between the visit status for the subject and visit records defined within the electronic data capture system. Identifying the visit alignment includes (a) determining whether the visit status is a scheduled visit and the electronic data capture system is ready to receive the health data; (b) determining whether the visit status is an unscheduled visit and the electronic data capture system is not ready to receive the health data; and (c) determining whether the visit status is an unscheduled visit and the visit status for the subject needs to be updated in the electronic data capture system. The DDC system identifies the visit alignment based on the determining in a, b, and/or c and determines the form based on the types of data and the visit alignment.

In some embodiments, the mapping involves identifying, by the DDC system, types of data within the health data and identifying a pre-built dataset matching template based on the type of data identified within the health data. The mapping also involves determining relationships between the data elements of the health data in the site specific format and the data elements of the form in the electronic data capture format using the pre-build dataset matching template. The DDC system matches the data elements of the health data in the site specific format to the counterpart data elements of the form in the electronic data capture format based on the relationships.

In some embodiments establishing instructions involves identifying, by the DDC system, types of data within the health data. The DDC system identifies whether the data translation between the data elements of the health data in the site specific format and the data elements of the health data in the electronic data capture format are to be constructive, destructive, aesthetic, structural, or a combination thereof based on the mappings, the types of data, and the form. Identifying the data translation includes (a) determining whether the data elements of the health data in the site specific format need to be translated between the site specific format and the electronic data capture format based on the mappings, the types of data, and the form; (b) determining whether the data elements of the health data in the site specific format need to be filtered, aggregated, summarized, or a combination thereof to fit within fields of the form; (c) determining whether the data elements of the health data in the site specific format need to be enriched or imputed based on business logic associated with the form; (d) determining whether the data elements of the health data in the site specific format need to be indexed or ordered based on the mappings, the types of data, and the form; (e) determining whether the data elements of the health data in the site specific format need to be encrypted based on the types data; and (f) determining whether the data elements of the health data in the site specific format need to be formatted, renamed, standardized, or a combination thereof based on the mappings, the types of data, and the form. The DDC system generates the instructions for how the data elements of the health data in the site specific format are to be translated to the data elements of the health data in the electronic data capture format based the determining in a, b, c, d, e, and/or f.

In some embodiments, translating the data elements involves selecting, by the DDC system, individual layers of processing for executing the data translating in accordance with the instructions. Each individual layer of processing is configured to perform a specific set of tasks that meet one or more of the translation processes determined in a, b, c, d, e, and/or f. The DDC system executes the instructions using the selected individual layers of processing to translate the data elements of the health data in the site specific format to the data elements of the health data in the electronic data capture format.

In some embodiments, the electronic data capture system is configured to generate the form based on the visit status of the subject and the information identifying the form for the subject, and populate fields of the form with the data elements of the health data in the electronic data capture format.

In some embodiments, the electronic data capture system is further configured to communicate the form with the data elements of the health data in the electronic data capture format to a data capture hub of an analysis system.

In some embodiments, the method further involves, prior to communicating to the electronic data capture system: displaying, by the DDC system, the data elements of the health data in the site specific format and the data elements of the health data in the electronic data capture format, in a user interface; receiving, by the DDC system, authorization from a user to communicate the data elements of the health data in the electronic data capture format to the electronic data capture system; and in response to receiving the authorization, communicating, by the DDC system, (i) the visit status of the subject, (ii) the information identifying the form for the subject, and (iii) the data elements of the health data in the electronic data capture format, to the electronic data capture system.

In some embodiments, the health data is structured data, unstructured data, or a combination thereof.

In some embodiments, the form is determined using one or more prediction models to predict the form based on the health data associated with the subject, and the visit status determined for the subject.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 depicts a block diagram of a computing environment for providing a direct data connection for universal collection of clinical trial health data according to various embodiments;

FIG. 2 depicts a block diagram of a process for providing a direct data connection for universal collection of clinical trial health data according to various embodiments;

FIGS. 3A-3D depict a user interface for universal collection of clinical trial health data according to various embodiments; and

FIG. 4 depicts a flowchart illustrating a process for universal collection of clinical trial health data using a direct data connection according to various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

The present disclosure describes techniques for translating health data from multiple formats into a standardized format. More specifically, embodiments of the present disclosure provide techniques for collecting health data in a site specific format and translating the health data, by a direct data connection (DDC) system, into an electronic data capture format.

Health data collected for various purposes such as a clinical health trial is typically collected and formatted based on the system collecting the data and the clinical site at which the data is collected. For example, an electronic health record (EHR) system may collect and format the health data in one way, whereas a clinical trial management system (CTMS) may collect and format the health data in a different way. However, traditional analysis methods for a clinical research coordinator of the clinical health trial involve the health data being in a particular format (referred to hereafter as an electronic data capture format). Traditional strategies for translating the health data into the electronic data capture format involve a human receiving the health data in the original format (e.g., recording the health data in an EHR, or collecting the health data on paper) and recreating the health data in the electronic data capture format. This typically involves the user manually copying the health data into the electronic data capture format, such as by typing the data manually into a computer while referencing paper notes or the health data in the EHR. Since the user manually transcribes the health data in the original format to generate the health data in the electronic data capture format, these traditional strategies are time consuming and error prone, which can lead to potential errors in the clinical health trial.

To address these limitations and problems, the techniques for health data translation in the present disclosure utilize an accurate and automated approach with minimal human interaction. Specifically, the techniques provide a direct data connection to the clinical sites such that data is coming directly from the source as it is captured through an automated interface not requiring human interaction to initiate the transfer. These techniques are intended to obtain, by the DDC system, health data in a site specific format, determine mappings between the site specific format and the electronic data capture format, and translate the health data into the electronic data capture format based on the mappings. This technique can also determine a visit status for the subject and a form for the subject that is to be completed that captures the health data. The health data may be displayed in the electronic data capture format on a user interface which a user can interact with to provide authorization that the health data is accurate. Then, the visit status, information identifying the form, and the data elements in the electronic data capture format can be communicated by the DDC system to an electronic data capture (EDC) system for further analysis.

One illustrative embodiment of the present disclosure is directed to a method that includes obtaining, by an application programming interface (API) of a DDC system, health data for a subject from a site. The health data is obtained in a site specific format. The method further includes determining, by the DDC system, a visit status of the subject associated with the health data. The visit status identifies whether the health data for the subject was collected in accordance with a scheduled visit or an unscheduled visit. The method further includes determining, by the DDC system, a form for the subject that is to be completed for capturing the health data based on: the health data associated with the subject, and the visit status determined for the subject. The method further includes mapping data elements of the health data in the site specific format to counterpart data elements of the form in an electronic data capture format; and establishing, by the DDC system, instructions for how the data elements of the health data in the site specific format are to be translated to the data elements of the health data in the electronic data capture format based on relationships inferred from the mappings. The method further includes translating, by the DDC system, the data elements of the health data in the site specific format to the data elements of the health data in the electronic data capture format based on the instructions; and communicating, by the DDC system, the visit status of the subject, information identifying the form for the subject, and the data elements of the health data in the electronic data capture format, to an electronic data capture system.

In some instances, the electronic data capture system is configured to generate the form based on the visit status of the subject and the information identifying the form for the subject, and populate the counterpart data elements of the form with the data elements of the health data in the electronic data capture format. The electronic data capture system is further configured to communicate the form with the data elements of the health data in the electronic data capture format to a data capture hub of an analysis system, and the analysis system is configured to analyze the data elements of the health data in the electronic data capture format with respect to a clinical trial protocol.

In some instances, the method further includes prior to communicating to the electronic data capture system, displaying, by the DDC system, the data elements of the health data in the site specific format and the data elements of the health data in the electronic data capture format, in a user interface. The method further includes receiving, by the DDC system, authorization from a user to communicate the data elements of the health data in the electronic data capture format to the electronic data capture system. In response to receiving the authorization, the method further includes communicating, by the DDC system, the visit status of the subject, the information identifying the form for the subject, and the data elements of the health data in the electronic data capture format, to the electronic data capture system.

### II. Example Computing Environment

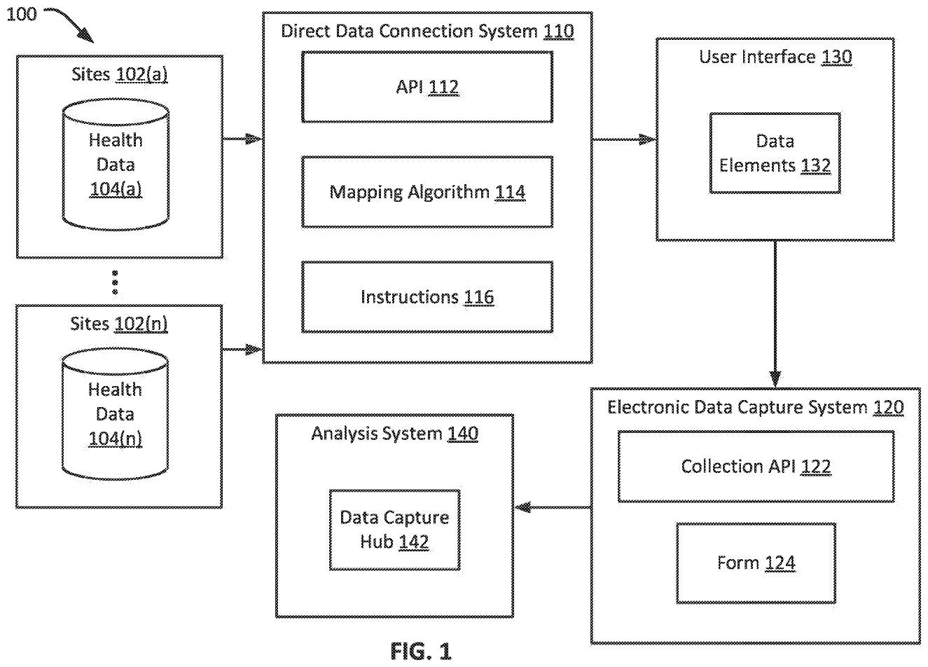

FIG. 1 depicts a block diagram of a computing environment 100 for providing a direct data connection for universal collection of clinical trial health data according to various embodiments. In the illustrated embodiment, computing environment 100 includes a direct data connection (DDC) system 110 in communication with sites 102a-n and a user interface 130. The components of the computing environment 100 may communicate via a network. For example, an API 112 can make a function call to an end point, such as site 102a, using a communication network. The communication network can be any type of network familiar to those skilled in the art that can support data communications using any of a variety of available protocols, including without limitation TCP/IP (transmission control protocol/Internet protocol), SNA (systems network architecture), IPX (Internet packet exchange), AppleTalk®, and the like. Merely by way of example, network(s) can be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

The sites 102a-n can collect health data 104a-n for subjects. The subjects can be enrolled in one or more clinical health trials and the health data 104a-n can be associated with the one or more clinical health trials. The health data 104a-n may include any data related to a physical state of a subject. For example, the health data 104a-n can include a height of a subject, a weight of the subject, and data related to laboratory tests performed on the subject (e.g., hematology test results, urinary test results, chemistry test results, etc.). The health data 104a-n may be deidentified at the sites 102a-n, such that each subject is associated with a subject identifier that cannot be traced to the subject. Additionally, the health data 104a-n may be structured data, unstructured data, or a combination thereof. Structured data is typically quantitative and searchable, while unstructured data is typically qualitative data stored in a native format.

In some instances, the DDC system 110 includes an application programming interface (API) 112 for obtaining health data for a subject. For example, the health data for the subject may be the health data 104a collected at sites 102a. Each of the sites 102a can be an endpoint of the API 112 and can include an electronic health record (EHR) system and/or a supplementary health data system (e.g., a data warehouse for a clinical health trial or a clinical trial management system (CTMS)) that each include the health data 104a for the subject. To obtain the health data 104a, the sites 102a can make a call using the API 112 to a server associated with the DDC system 110. The sites 102a can locate the health data 104 for the subject and automatically push the health data 104a to the DDC system 110. The DDC system 110 may alternatively pull the health data 104a using the API 112. The health data 104a may be obtained in site specific formats. For example, health data from an EHR may be in a first site specific format and health data from a CTMS may be in a different site specific format.

In some instances, the DDC system 110 determines a visit status of the subject associated with the health data 104a. The visit status can identify whether the health data 104a for the subject was collected in accordance with a scheduled visit or an unscheduled visit. A scheduled visit is a collection of health data that was planned prior to the collection. Alternatively, an unscheduled visit is a collection of health data that was not planned prior to the collection. The DDC system 110 can determine the visit status based on an input received from a user or based on known visit dates. If the visit date associated with the health data 104a-n does not match any of the known visit dates, the DDC system 110 can determine the visit status is an unscheduled visit.

The DDC system 110 determines a form 124 for the subject that is to be completed for capturing the health data 104a. The DDC system 110 can determine the form 124 based on the health data 104a associated with the subject and the visit status determined for the subject. For example, the health data 104a including certain types of data (e.g., sodium levels, potassium levels, blood cell count, etc.) can be associated with a particular form. The DDC system 110 can determine types of data included in the health data 104a and determine the form 124 based on the types of data.

A mapping algorithm 114 of the DDC system 110 maps data elements of the health data 104a in the site specific format to counterpart data elements of the form 124 in an electronic data capture format. The electronic data capture format can be a standardized format that any health data 104a-n from the sites 102a-n can be translated into. The mapping algorithm 114 can determine how a data element of the health data 104a in the site specific format relates to a data element in the electronic data capture format. The mapping algorithm 114 may use natural language processing or other techniques to determine the relationships. Based on the determined relationship, the mapping algorithm 114 can establish instructions 116 for how the data elements of the health data 104a in the site specific format are to be translated to the data elements of the health data 104a in the electronic data capture format. For example, the mapping algorithm 114 may determine that health data related to a body weight is expressed as “bdy wt” in the site specific format and as “body weight” in the electronic data capture format. As a result, the instructions 116 can specify that data elements received as “bdy wt” in the site specific format are to be translated to data elements of “body weight” in the electronic data capture format.

In some instances, the DDC system 110 translates the data elements of the health data 104a in the site specific format to the data elements of the health data 104a in the electronic data capture format based on the instructions 116. To illustrate, the DDC system 110 can translate the data elements of “bdy wt” in the site specific format to the data elements of “body weight” in the electronic data capture format.

The data elements in the site specific format and the electronic data capture format may be presented in a user interface 130 as data elements 132. The user interface 130 can be used by a user to verify accuracy of the translation of the data elements 132 from the site specific format to the electronic data capture format. The user can give authorization to the DDC system 110 via the user interface 130 to communicate the data elements 132 of the health data 104a in the electronic data capture format to an electronic data capture (EDC) system 120. In response to receiving the authorization, the DDC system 110 can communicate the visit status of the subject, information identifying the form 124 for the subject, and the data elements 132 of the health data 104a in the electronic data capture format to the EDC system 120. A collection API 122 of the EDC system 120 can receive the communicated information from the DDC system 110.

In some instances, the EDC system 120 generates the form 124 with the data elements 132 of the health data 104a in the electronic data capture format. The EDC system 120 can generate the form 124 based on the visit status of the subject and the information identifying the form 124 for the subject. For example, the EDC system 120 can determine the visit status and the information identifying the form 124 correspond to an unscheduled visit urinalysis form. As a result, the form 124 generated by the EDC system 120 can be the unscheduled visit urinalysis form. The EDC system 120 can populate the counterpart data elements of the form 124 with the data elements 132 of the health data 104a in the electronic data capture format. Thus, the form 124 can be standardized such that each form generated of a particular type (e.g., unscheduled visit urinalysis form) is in the same format and includes the same information except for the particular health data values that are populated.

The DDC system 110 works with the EDC system 120 to seamlessly execute downstream processes. The DDC system 110 can integrate with the EDC system 120 to determine patient-to-visit alignment, data entry and viewing permissions, form-to-field mappings, as well as controlled vocabularies for each field. The DDC system 110 can interpret a clinical health trial workflow from the EDC system 120 and mimic it to stay in sync with the clinical health trial design and adaptations over time. The EDC system 120 can also communicate the form 124 to a data capture hub 142 of an analysis system 140. The data capture hub 142 can include a collection of forms for one or more clinical health trials. For example, forms associated with the clinical health trial that the subject is enrolled in can be stored in the data capture hub 142. The analysis system 140 can be associated with a physician or other clinical research coordinator of the clinical health trial. The clinical research coordinator of the clinical health trial can analyze the forms in the data capture hub 142 to determine a change to be implemented in the clinical health trial (e.g., an adjustment to a treatment) and/or an outcome of the clinical health trial.

### III. Techniques for Health Data Collection and Translation

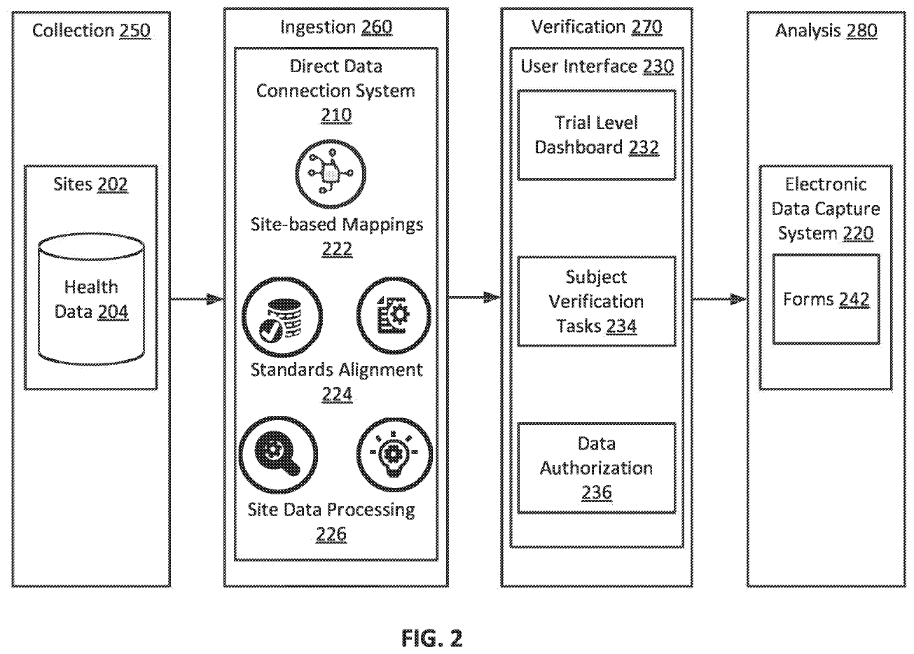

FIG. 2 depicts a block diagram of a process for providing a direct data connection for universal collection of clinical trial health data according to various embodiments. The process can be implemented by the systems in FIG. 1. For example, FIG. 2 includes a DDC system 210, a user interface 230, and an EDC system 220, which correspond to the DDC system 110, the user interface 130, and the EDC system 120, respectively.

During a collection step 250, health data 204 is obtained at sites 202. The sites 202 can include an EHR system, a CTMS, and/or other supplementary health data systems. The sites 202 can be associated with a clinical health trial and obtain the health data 204 from subject(s) enrolled in the clinical health trial. Each of the sites 202 can obtain the data in a site specific format that varies between the sites 202. Additionally, the health data 204 collected by the sites 202 may be structured data, unstructured data, or a combination thereof.

During an ingestion step 260, the DDC system 210 obtains the health data 204 from the sites 202. The DDC system 210 can determine site-based mappings 222 for the health data 204. The site-based mappings 222 can map the health data 204 in the site specific formats to an electronic data capture format. To determine the site-based mappings 222, the DDC system 210 can identify types of data (e.g., sodium value, potassium value, height, weight, blood pressure, etc.) within the health data 204. The DDC system 210 can then identify a pre-built dataset matching template based on the type of data identified. The DDC system 210 uses the pre-built dataset matching template to determine relationships between the data elements of the health data in the site specific format and the data elements of the form in the electronic data capture format. For example, the pre-built dataset matching template may indicate that a “sodium value” data element in the site specific format matches a “Na value” data element in the electronic data capture format. The DDC system 210 can then match the data elements in the site specific format to the counterpart data elements in the electronic data capture format based on the relationships.

The DDC system 210 can also perform standards alignment 224 to ensure the health data 204 in the electronic data capture format meets certain standards and regulations (e.g., deidentification standards). Standards alignment 224 may be part of establishing instructions for how to translate the data elements in the site specific format to the data elements in the electronic data capture format. For example, establishing the instructions can involve the DDC system 210 determining whether the data elements of the health data 204 in the site specific format encrypted based on the type of data and any standards or regulations. Standards alignment 224 may additionally involve the DDC system 210 establishing instructions for standardizing the data elements in the site specific format.

The DDC system 210 also performs site data processing 226, which can include determining a visit status (e.g., scheduled visit or unscheduled visit) of the subject associated with the health data 204. To determine the visit status, the DDC system 210 can identify an actual visit date on which the health data 204 was collected and identify whether a scheduled visit date exists for the subject on the actual visit date. The actual visit date can be determined based on source data at the sites 202 and metadata associated with the health data 204. For example, each data point of the health data 204 can include an associated date for the actual visit date that the data point was collected. The DDC system 210 can receive a predefined list of scheduled visit dates for the subject from the EDC system 220 and determine, based on comparing the actual visit date to the predefined list of scheduled visit dates, whether the visit status is a scheduled visit or an unscheduled visit. The DDC system 210 can determine the visit status as an unscheduled visit if the scheduled visit date does not exist for the actual visit date and the DDC system 210 can determine the visit status as a scheduled visit if the scheduled visit date exists for the subject on the actual visit date.

The site data processing 226 can also include determining a form for the subject that is to be completed for capturing the health data 204 based on the health data 204 associated with the subject and the visit status. Determining the form can include the DDC system 210 identifying types of data within the health data 204. The types of data can be determined from a study protocol and an electronic case report form for the clinical health trial. The DDC system 210 can include a mapping table specifying which data elements and types of data belong to each form. The DDC system 210 can identify a visit alignment between the visit status for the subject and visit records defined within the EDC system 220. For example, identifying the visit alignment can include determining whether the visit status is a scheduled visit and the EDC system 220 is ready to receive the health data 204 (e.g., the EDC system 220 does not include a visit record for the visit date). The subject can be associated with a clinical trial arm in the EDC system 220. The clinical trial arm can be aligned to possible visit dates that the subject can be assigned to. The EDC system 220 can be ready to receive health data 204 if the visit date is one of the possible visit dates for the subject. There can be certain data elements that are to be collected at each visit. A subset of visits that a subject can provide data for can then be determined along with the types of data that are to be collected at each visit. Based on this, the DDC system 210 can determine a range of available visits for each data form to be presented. A user can select the visit date from the available visits. Identifying the visit alignment can also include the DDC system 210 determining whether the visit status is an unscheduled visit and the EDC system 220 is not ready to receive the health data 204 (e.g., the EDC system 220 does not include a visit record for the visit date), or determining whether the visit status is an unscheduled visit and the visit status for the subject needs to be updated in the EDC system 220 (e.g., to include a visit record for the visit date). The DDC system 210 can present the available visits and the user can select an unscheduled visit option if the data collection does not align to one of the available visits. The DDC system 210 may alternatively transmit the health data 204 of an unscheduled visit to the EDC system 220, and the EDC system 220 can automatically generate the visit. Once the visit alignment is determined, the DDC system 210 can determine the form based on the types of data and the visit alignment. The form may be an unscheduled chemistry local form, a scheduled chemistry local form, an unscheduled hematology form, a scheduled hematology form, etc. In some instances, the DDC system 210 may use one or more prediction models to predict the form based on the health data 204 and the visit status. The one or more prediction models may predict the form based on the visit date associated with the health data 204 and a schedule of assessments from a protocol of the clinical health trial.

Site data processing 226 may also involve establishing instructions for how the data elements of the health data 204 in the site specific format are to be translated into the electronic data capture format. The instructions can be based on the site-based mappings 222. For example, the DDC system 210 can identify the types of data within the health data 204 and identify whether the data translation between data elements in the site specific format and data elements in the electronic data capture format are to be constructive, destructive, aesthetic, structural, or a combination thereof based on the site-based mappings 222. The types of data can be defined by the forms that contain the data fields. For example, if a data element corresponds to a Red Blood Cell count (RBC), then RBC is a data field, but the form that the data field is collected on can be a hematology form. The translation can be constructive if the data elements in the site specific format are to be translated between the site specific format and the electronic data capture format based on the site-based mappings 222, the types of data, and the form. The translation can be destructive if the data elements in the site specific format are to be filtered, aggregated, summarized, or a combination thereof to fit within fields of the form. The translation can be aesthetic if the data elements in the site specific format are to be enriched or imputed based on business logic associated with the form. Enriching data elements can involve changing units of a measurement. The translation can be structural if the data elements in the site specific format are to be indexed or ordered based on the site-based mappings 222, the types of data, and the form. Establishing the instructions can also include the standards alignment 224, where the translation can involve encrypting data elements based on the types of data, or determining whether data elements in the site specific format are to be formatted, renamed, standardized, or a combination thereof based on the site-based mappings 222, the types of data, and the form. The instructions can be generated based on the determination of how the data elements are to be translated.

The DDC system 210 can then translate the health data 204 in the site specific format into the electronic data capture format using the instructions during the site data processing 226. The DDC system 210 can select individual layers of processing for executing the data translation in accordance with the instructions. Each individual layer of processing can be configured to perform a specific set of tasks that meet one or more of the translate processes (e.g., constructive transformation, destructive transformation, aesthetic transformation, and structural transformation). The DDC system 210 can execute the instructions using the selected layer of processing to translate the data elements of the health data 204 in the site specific format to the data elements in the electronic data capture format.

During a verification step 270, the user interface 230 can include a trial level dashboard 232. The trial level dashboard 232 can show information about the clinical health trial. For example, the trial level dashboard 232 can show all subjects associated with the clinical health trial and information related to each subject (e.g., a number of forms to be completed). Access to the trial level dashboard 232 may be controlled for each user. For example, each user may only have access to a trial level dashboard for which they are a clinical research coordinator or a data coordinator.

The user can navigate within the trial level dashboard 232 to submit health data 204 for the subjects. For example, the user can perform subject verification tasks 234 such as selecting a particular subject of a study and verifying health data 204 from the sites 202 corresponds to the particular subject. The user may also select a visit date for the health data 204. The health data 204 for the determined form for the subject can be populated on the user interface 230 in the electronic data capture format. The user can then perform data authorization 236 to verify the data is accurate relative to the health data 204 from the sites 202. The data authorization 236 can involve the user making a selection on the user interface to transmit the health data 204 in the electronic data capture format to the EDC system 220.

During an analysis step 280, forms 242 that include the health data 204 in the electronic data capture format can be generated and analyzed in the EDC system 220. An API of the EDC system 220 can determine whether the forms 242 exists for the health data 204. If the forms 242 do not exist, the EDC system 220 can generate the forms 242. In some instances, the forms 242 can be transmitted to a data capture hub of an analysis system (e.g., data capture hub 142 of the analysis system 140). A clinical research coordinator of the clinical health trial (or another person associated with the clinical health trial) can then analyze the forms 242 with respect to a clinical trial protocol (e.g., a dosage of a medication, a type of treatment, etc.). The clinical research coordinator may then adjust the clinical health trial based on the analysis of the forms 242 in the electronic data capture format. Alternatively, the analysis step 280 may be performed by a computing device. The computing device may include a machine-learning model configured to make inferences based on the health data 204. The computing device can output the inferences to the clinical research coordinator or other individual.

### IV. User Interface

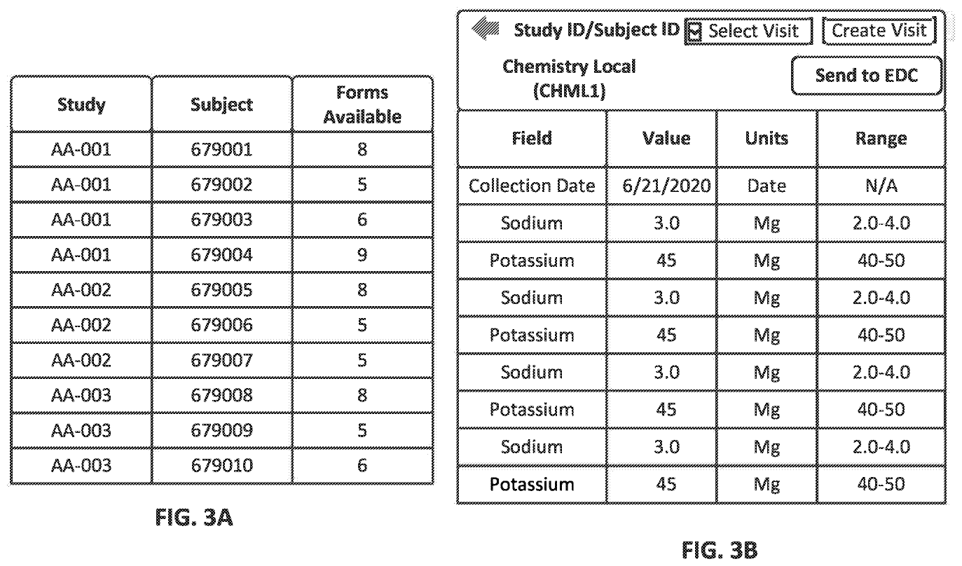

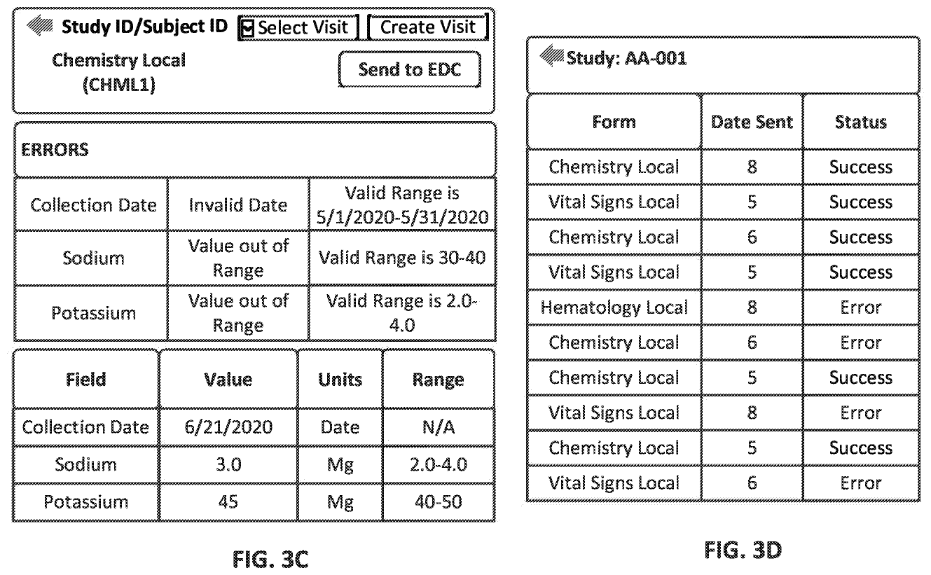

FIGS. 3A-3D depict a user interface for universal collection of clinical trial health data according to various embodiments. The user interface may be in connection with a DDC system (e.g., DDC system 110 or DDC system 210) and an EDC system (e.g., EDC system 120 or EDC system 220).

FIG. 3A depicts an example of the user interface showing an overview page for clinical health trials. Each of the clinical health trials may be associated with a same entity (e.g., organization or clinical research coordinator). The user interface can show health data collected from one or more sites and present the health data to a user to be authorized for sending to the EDC system. As illustrated, the user interface shows three clinical health trials: AA-001, AA-002, and AA-003. Subjects can be associated with each of the clinical health trials for which forms are collected. FIG. 3A shows clinical health trial AA-001 as including subjects 679001, 679002, 679003, and 679004. The identity of the subject is not presented to the user, as each subject has been deidentified and is only addressed by their subject identification number. Each of the subjects have a differing number of forms available to be completed. For example, subject 679001 has eight forms available to be completed. These forms can be any combination of a urinalysis form, a microurinalysis form, a hematology form, a chemistry form, or any other suitable form. The forms may be for a particular visit date that the health data was to be collected for the subject.

FIG. 3B depicts an example of the user interface after a particular study and subject identification have been selected. As illustrated, health data related to sodium levels and potassium levels were collected for the particular subject enrolled in the clinical health trial on the data Jun. 21, 2020. The user has the option to select a visit date or create a visit. In some instances, the DDC system determines possible visit dates and the user can select the actual visit date from the possible visit dates. The user may alternatively select that the health data corresponds to an unscheduled visit, where a visit is to be created for the health data.

The health data is presented on the user interface in an electronic data capture format. The DDC system can determine mappings from the site specific format to the electronic data capture format, establish instructions for how to map the data elements from the former format to the later format, and translate the data elements into the electronic data capture format based on the instructions. The DDC system then displays the data elements in the electronic data capture format, as illustrated in FIG. 3B. The user can verify the accuracy of the populated data without manually entering the data elements. Then the user can select the “send to EDC” user interface feature to give authorization to communicate the data elements in the electronic data capture format to the EDC system.

In response to receiving the input from the user of the selection of the “send to EDC” user interface feature, the DDC system can communicate the visit status of the subject (e.g., scheduled visit or unscheduled visit) the information identifying the form (e.g., chemistry local), and the data elements in the electronic data capture format to the EDC system. The EDC system can then generate a form for the subject in the electronic data capture format based on the received communication. Additionally, the EDC system can communicate the form to an analysis system to facilitate analysis of the health data with respect to a clinical trial protocol.

FIG. 3C depicts an example of the user interface subsequent to the user authorizing the health data to be communicated to the EDC system. As illustrated, the user interface may present errors based on the data elements in the electronica data capture format. The DDC system can determine the errors and display them on the user interface. For example, the DDC system can include a predefined range of values for each data element. Upon determining a data element is outside of the predefined range, the DDC system can determine the value includes an error and display the error. FIG. 3C includes three errors: one for the collection date, one for the sodium level, and one for the potassium level. Each of the errors corresponds to a value outside of a predefined range. The user interface may additionally include a display of the valid, predefined range for each data element. For example, the valid range for the collection date is May 1, 2020 to May 31, 2020, the valid range for the sodium value is 30 mg to 40 mg and the valid range for the potassium value is 2.0 mg to 4.0 mg. The user interface may also include additional information related to the errors or missing information, although not shown in FIG. 3C. The DDC system may not mitigate errors, but may indicate to a site where the health data was collected than there is an error so that the errors can be corrected at the site (e.g., within the CTMS, LIMS, etc.).

FIG. 3D depicts the user interface showing forms of health data collected in the electronic data capture format for clinical health trial AA-001. Each form may be associated with the same or different subjects. The forms generated include chemistry local forms, vital signs local forms, and hematology local forms. The user interface also includes the date that each form was sent to the EDC system. Each date is represented as a numerical value (e.g., 6, 5, and 8), but they could alternatively be shown as the date written out (e.g., May 31, 2020). Additionally, the user interface can include an indication of whether the data elements in each form were successfully communicated to the EDC system, or whether errors, such as those shown in FIG. 3C occurred. An indication of an error can indicate that the user (or a health data collector at the site) should review the health data collected for the corresponding visit in the source database to determine how the error was introduced. A full log of transactions and health data submissions can be stored and may be displayed on the user interface. The log can indicate what health data was submitted, which user submitted the health data, and when the submission occurred.

### V. Example Methods

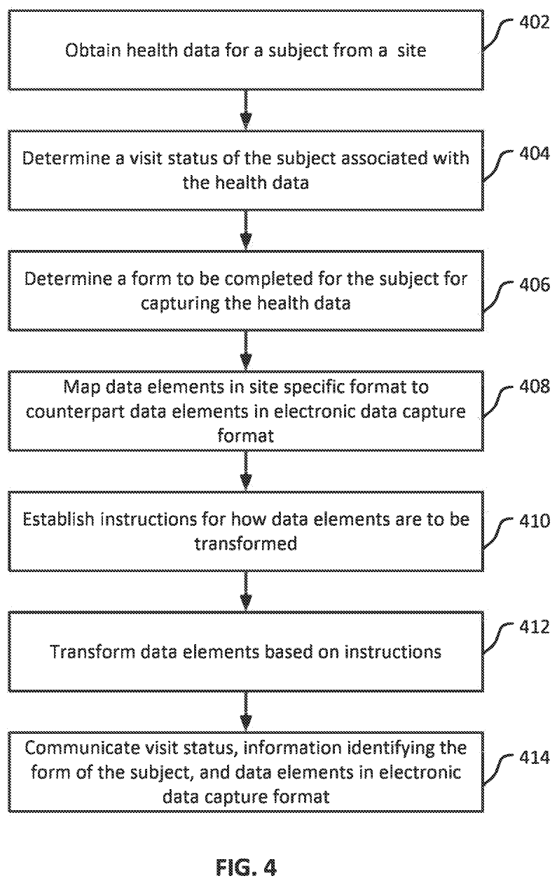

FIG. 4 depicts a flowchart illustrating a process 400 for universal collection of clinical trial health data using a direct data connection according to various embodiments. The processes depicted in process 400 are implemented by the architecture, systems, and techniques depicted in FIGS. 1, 2 and 3A-3D. Individual embodiments may be described as a process which is depicted as a flowchart, a flow diagram, a data flow diagram, a structure diagram, or a block diagram. Although a flowchart may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

The processes and/or operations depicted in FIG. 4 may be implemented in software (e.g., code, instructions, program) executed by one or more processing units (e.g., processors cores), hardware, or combinations thereof. The software may be stored in a memory (e.g., on a memory device, on a non-transitory computer-readable storage medium). The particular series of processing steps in FIG. 4 is not intended to be limiting. Other sequences of steps may also be performed according to alternative embodiments. For example, in alternative embodiments the steps outlined above may be performed in a different order. Moreover, the individual steps illustrated in FIG. 4 may include multiple sub-steps that may be performed in various sequences as appropriate to the individual step. Furthermore, additional steps may be added or removed depending on the particular applications. One of ordinary skill in the art would recognize many variations, modifications, and alternatives.

At block 402, health data is obtained for a subject from a site (e.g., EHR system, CTMS system, data warehouse, etc.). The health data can be obtained by a DDC system, such as those described in FIGS. 1 and 2. The site can be a collection site for health data related to a clinical health trial for which the subject is enrolled. The health data may be in a site specific format and the site specific format for each site may differ from the site specific format of other sites. The health data may be unstructured data, structured data, or a combination thereof. The health data can additionally be deidentified prior to being obtained.

At block 404, a visit status of the subject associated with the health data is determined. The DDC system can determine the visit status to be a scheduled visit if the health data was expected to be collected on the visit date prior to the subject visiting. If the health data was not expected to be collected on the visit date prior to the subject visiting, the DDC system can determine the visit status to be an unscheduled visit based on a selection from a user.

At block 406, a form to be completed for the subject is determined. The form corresponds to the health data associated with the subject and the visit status determined for the subject. The DDC system can determine a type of data (e.g., sodium value, potassium value, height, weight, blood pressure, etc.) for each data element of the health data and use the determined type of data in conjunction with the visit status determined for the subject to ultimately determine the form. For example, the DDC system may determine the form to be a chemistry form for a scheduled visit if the health data includes sodium values and potassium values and the visit status is determined to be a scheduled visit.

At block 408, data elements in the site specific format are mapped to counterpart data elements of the form in an electronic data capture format. The electronic data capture format can be a standardized format for representing the health data of any site specific format. The DDC system can determine data elements in the electronic data capture format that relate to each data element in the site specific format and generate the mapping based on the determination.

At block 410, instructions are established for how the data elements are to be translated. The instructions are based on the mappings. For example, an instruction can specify data elements corresponding to a height value represented as “height” in a site specific format are to be translated to data elements represented as “ht” in the electronic data capture format based on a mapping between the “height” data element and the “ht” data element. In some instances, the instruction may further specify a data translation for standardizing a value for the data elements. For example, an instruction can specify a formula for translating the “height” data element in meters to the “ht” data element in feet.

At block 412, the data elements of the health data in the site specific format are translated to data elements of the health data in the electronic data capture format. The DDC system translates the data elements according to the instructions to generate the data elements in the electronic data capture format. The data elements in the electronic data capture format may then be displayed on a user interface, such that authorization can be received from a user to communicate the data elements to an EDC system.

At block 414, the visit status, information identifying the form of the subject, and the data elements in the electronic data capture format are communicated to the EDC system. The communication can happen in response to the DDC system receiving the authorization from the user. The EDC system can generate the form with the data elements in the electronic data capture based on the received communication. Additionally, the EDC system can communicate the form to an analysis system to facilitate analysis of the health data with respect to a clinical trial protocol.

### VI. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
obtaining, by an application programming interface (API) of a direct data connection (DDC) system, health data for a subject from a site, wherein the health data is obtained in a site specific format;
determining, by the DDC system, a visit status of the subject associated with the health data, wherein the visit status identifies whether the health data for the subject was collected in accordance with a scheduled visit or an unscheduled visit;
determining, by the DDC system, a form for the subject that is to be completed for capturing the health data based on: (i) the health data associated with the subject, and (ii) the visit status determined for the subject;
mapping, by the DDC system, data elements of the health data in the site specific format to counterpart data elements of the form in an electronic data capture format;
establishing, by the DDC system, instructions for how the data elements of the health data in the site specific format are to be translated to the data elements of the health data in the electronic data capture format based on the mappings;
translating, by the DDC system, the data elements of the health data in the site specific format to the data elements of the health data in the electronic data capture format based on the instructions; and
communicating, by the DDC system, (i) the visit status of the subject, (ii) information identifying the form for the subject, and (iii) the data elements of the health data in the electronic data capture format, to an electronic data capture system.

2. The computer-implemented method of claim 1, wherein the health data is obtained from a plurality of sites including an electronic health record (EHR) system and a supplementary health data system, and the health data is obtained in a plurality of different site specific formats.

3. The computer-implemented method of claim 1, wherein the health data is obtained by the site making a call using the API to a server associated with DDC system.

4. The computer-implemented method of claim 1, wherein the determining the visit status comprises:
identifying, by the DDC system, an actual visit date on which the health data was collected;
identifying, by the DDC system, whether a scheduled visit date exists for the subject on the actual visit date;
in response to identifying the scheduled visit date exists for the subject on the actual visit date, determining, by the DDC system, the visit status as a scheduled visit; and
in response to identifying the scheduled visit date does not exist for the subject on the actual visit date, determining, by the DDC system, the visit status as an unscheduled visit.

5. The computer-implemented method of claim 1, wherein the determining the form comprises:
identifying, by the DDC system, types of data within the health data;
identifying, by the DDC system, a visit alignment between the visit status for the subject and visit records defined within the electronic data capture system, wherein the identifying the visit alignment comprises:
(a) determining whether the visit status is a scheduled visit and the electronic data capture system is ready to receive the health data;
(b) determining whether the visit status is an unscheduled visit and the electronic data capture system is not ready to receive the health data;
(c) determining whether the visit status is an unscheduled visit and the visit status for the subject needs to be updated in the electronic data capture system; and
identifying the visit alignment based on the determining in a, b, and/or c; and determining, by the DDC system, the form based on the types of data and the visit alignment.

6. The computer-implemented method of claim 1, wherein the mapping comprises:
identifying, by the DDC system, types of data within the health data;
identifying, by the DDC system, a pre-built dataset matching template based on the type of data identified within the health data;
determining, by the DDC system using the pre-built dataset matching template, relationships between the data elements of the health data in the site specific format and the data elements of the form in the electronic data capture format; and
matching, by the DDC system, the data elements of the health data in the site specific format to the counterpart data elements of the form in the electronic data capture format based on the relationships.

7. The computer-implemented method of claim 1, wherein the establishing instructions comprises:
identifying, by the DDC system, types of data within the health data;
identifying, by the DDC system, whether the data translation between the data elements of the health data in the site specific format and the data elements of the health data in the electronic data capture format are to be constructive, destructive, aesthetic, structural, or a combination thereof based on the mappings, the types of data, and the form, wherein the identifying the data translation comprises:
(a) determining whether the data elements of the health data in the site specific format need to be translated between the site specific format and the electronic data capture format based on the mappings, the types of data, and the form;
(b) determining whether the data elements of the health data in the site specific format need to be filtered, aggregated, summarized, or a combination thereof to fit within fields of the form;
(c) determining whether the data elements of the health data in the site specific format need to be enriched or imputed based on business logic associated with the form;
(d) determining whether the data elements of the health data in the site specific format need to be indexed or ordered based on the mappings, the types of data, and the form;
(e) determining whether the data elements of the health data in the site specific format need to be encrypted based on the types of data; and
(f) determining whether the data elements of the health data in the site specific format need to be formatted, renamed, standardized, or a combination thereof based on the mappings, the types of data, and the form; and
generating the instructions for how the data elements of the health data in the site specific format are to be translated to the data elements of the health data in the electronic data capture format based the determining in a, b, c, d, e, and/or f.

8. The computer-implemented method of claim 7, wherein the translating the data elements comprises:
selecting, by the DDC system, individual layers of processing for executing the data translation in accordance with the instructions, wherein each individual layer of processing is configured to perform a specific set of tasks that meet one or more of the translation processes determined in a, b, c, d, e, and/or f; and
executing, by the DDC system using the selected individual layers of processing, the instructions to translate the data elements of the health data in the site specific format to the data elements of the health data in the electronic data capture format.

9. The computer-implemented method of claim 1, wherein the electronic data capture system is configured to generate the form based on the visit status of the subject and the information identifying the form for the subject, and populate fields of the form with the data elements of the health data in the electronic data capture format.

10. The computer-implemented method of claim 9, wherein the electronic data capture system is further configured to communicate the form with the data elements of the health data in the electronic data capture format to a data capture hub of an analysis system.

11. The computer-implemented method of claim 1, further comprising, prior to communicating to the electronic data capture system:
displaying, by the DDC system, (i) the data elements of the health data in the site specific format and (ii) the data elements of the health data in the electronic data capture format, in a user interface;
receiving, by the DDC system, authorization from a user to communicate the data elements of the health data in the electronic data capture format to the electronic data capture system; and
in response to receiving the authorization, communicating, by the DDC system, (i) the visit status of the subject, (ii) the information identifying the form for the subject, and (iii) the data elements of the health data in the electronic data capture format, to the electronic data capture system.

12. The computer-implemented method of claim 1, wherein the health data is structured data, unstructured data, or a combination thereof.

13. The computer-implemented method of claim 1, wherein the form is determined using a one or more prediction models to predict the form based on: (i) the health data associated with the subject, and (ii) the visit status determined for the subject.

14. A system comprising:
one or more data processors; and
a non-transitory computer readable storage medium containing instructions which, when executed on the one or more data processors, cause the one or more data processors to perform operations comprising:
obtaining, by an application programming interface (API) of a direct data connection (DDC) system, health data for a subject from a site, wherein the health data is obtained in a site specific format;
determining, by the DDC system, a visit status of the subject associated with the health data, wherein the visit status identifies whether the health data for the subject was collected in accordance with a scheduled visit or an unscheduled visit;
determining, by the DDC system, a form for the subject that is to be completed for capturing the health data based on: (i) the health data associated with the subject, and (ii) the visit status determined for the subject;
mapping, by the DDC system, data elements of the health data in the site specific format to counterpart data elements of the form in an electronic data capture format;
establishing, by the DDC system, instructions for how the data elements of the health data in the site specific format are to be translated to the data elements of the health data in the electronic data capture format based on the mappings;
translating, by the DDC system, the data elements of the health data in the site specific format to the data elements of the health data in the electronic data capture format based on the instructions; and
communicating, by the DDC system, (i) the visit status of the subject, (ii) information identifying the form for the subject, and (iii) the data elements of the health data in the electronic data capture format, to an electronic data capture system.

15. The system of claim 14, wherein the health data is obtained from a plurality of sites including an electronic health record (EHR) system and a supplementary health data system, and the health data is obtained in a plurality of different site specific formats.

16. The system of claim 14, wherein the health data is obtained by the site making a call using the API to a server associated with DDC system.

17. The system of claim 14, wherein the determining the visit status comprises:
identifying, by the DDC system, an actual visit date on which the health data was collected;
identifying, by the DDC system, whether a scheduled visit date exists for the subject on the actual visit date;
in response to identifying the scheduled visit date exists for the subject on the actual visit date, determining, by the DDC system, the visit status as a scheduled visit; and
in response to identifying the scheduled visit date does not exist for the subject on the actual visit date, determining, by the DDC system, the visit status as an unscheduled visit.

18. The system of claim 14, wherein the determining the form comprises:
identifying, by the DDC system, types of data within the health data;
identifying, by the DDC system, a visit alignment between the visit status for the subject and visit records defined within the electronic data capture system, wherein the identifying the visit alignment comprises:
(a) determining whether the visit status is a scheduled visit and the electronic data capture system is ready to receive the health data;
(b) determining whether the visit status is an unscheduled visit and the electronic data capture system is not ready to receive the health data;
(c) determining whether the visit status is an unscheduled visit and the visit status for the subject needs to be updated in the electronic data capture system; and
identifying the visit alignment based on the determining in a, b, and/or c; and determining, by the DDC system, the form based on the types of data and the visit alignment.

19. The system of claim 14, wherein the mapping comprises:
identifying, by the DDC system, types of data within the health data;
identifying, by the DDC system, a pre-built dataset matching template based on the type of data identified within the health data;
determining, by the DDC system using the pre-built dataset matching template, relationships between the data elements of the health data in the site specific format and the data elements of the form in the electronic data capture format; and
matching, by the DDC system, the data elements of the health data in the site specific format to the counterpart data elements of the form in the electronic data capture format based on the relationships.

20. A computer-program product tangibly embodied in a non-transitory machine-readable storage medium, including instructions configured to cause one or more data processors to perform operations comprising:
obtaining, by an application programming interface (API) of a direct data connection (DDC) system, health data for a subject from a site, wherein the health data is obtained in a site specific format;
determining, by the DDC system, a visit status of the subject associated with the health data, wherein the visit status identifies whether the health data for the subject was collected in accordance with a scheduled visit or an unscheduled visit;
determining, by the DDC system, a form for the subject that is to be completed for capturing the health data based on: (i) the health data associated with the subject, and (ii) the visit status determined for the subject;
mapping, by the DDC system, data elements of the health data in the site specific format to counterpart data elements of the form in an electronic data capture format;
establishing, by the DDC system, instructions for how the data elements of the health data in the site specific format are to be translated to the data elements of the health data in the electronic data capture format based on the mappings;
translating, by the DDC system, the data elements of the health data in the site specific format to the data elements of the health data in the electronic data capture format based on the instructions; and
communicating, by the DDC system, (i) the visit status of the subject, (ii) information identifying the form for the subject, and (iii) the data elements of the health data in the electronic data capture format, to an electronic data capture system.
